(19) United States
(12) Patent Application Publication
(10) Pub. No.: US 2003/0212425 A1
(43) Pub. Date: Nov. 13, 2003

(54) Device for assisting laparoscopic surgery

(76) Inventor: Anatole Menon-Johansson, London (GB)

Correspondence Address: William M. Lee, Jr., Lee, Mann, Smith, McWilliams, Sweeney & Ohlson, P.O.Box 2786, Chicago, IL, 60690-2786, US

(21) Appl. No.: 10/141,360

(22) Filed: May 8, 2002

(51) Int. Cl. A61B 17/34

(52) U.S. Cl. 606/185

(57) ABSTRACT

A tool is described which is adapted to be attached to the foremost end of a laparoscopic instrument. The tool has a ring at its foremost end adapted to be pushed into contact with the abdomen wall at a point where a trocar is to be inserted, for supporting the abdominal wall around the entry point of the trocar. To assist its use the plane of the ring is angled relative to the lengthwise axis of the instrument, and advantageously the diameter of the ring is selected so that it will admit the tip of the trocar but is too small to allow the trocar to pass completely therethrough. Thus by pushing the ring against the peritoneum, the trocar will be prevented from penetrating significantly into the abdominal cavity. A clamp or stop prevents the tool from leaving the instrument. The instrument may be a laparoscopic telescope, or a probe which is inserted through another incision for viewing by a telescope. A method of controlling the insertion of a trocar through the abdomen of a patient is described using a laparoscopic instrument fitted with such a tool wherein the trocar is first pushed gently against the abdomen where an incision is to be made, the region is viewed from within using a laparoscopic telescope, the ring is positioned against the peritoneum so that the ring surrounds the protrusion in the peritoneum caused by the pressure of the trocar against the external surface of the abdomen wall, and thereafter the pressure of the trocar on the skin is increased and the trocar rotated in known manner to form an incision, whilst firmly pressing the ring in the opposite sense to resist inward movement of the trocar beyond that permitted by the ring. Typically the tool is mounted on the telescope and the combination is pushed through a first incision and used to view and support the peritoneum, while a second incision is made by the trocar.

### FIELD OF THE INVENTION

This invention concerns a device and method to assist in the insertion of laparoscopic trocars.

### BACKGROUND OF THE INVENTION

Minimally invasive surgery creates significant benefits for the patient and the health care delivery service. Patients have fewer post-operative complications plus a speedier discharge following such procedures, whilst hospitals are able to safely increase their turnover of patients because of these shorter hospital stays.

Laparoscopic surgery is being used to treat an increasing number of conditions in a range of specialties, including (Gynaecology, General surgery, Cardio-thoracic surgery and Orthopaedics.

Laparoscopie surgery involves the following stages:

Anaesthetise the patient

Umbilical tool and then port insertion

Insufflation of the abdomen with gas or physically lifting up the abdominal wall

Insertion of telescope

Insertion of further abdominal tools

Performance of surgical procedure

Removal of tools

Suturing of abdominal wounds

The least elegant part of laparoscopic procedures is the insertion of second and subsequent abdominal tools. These have to be preceded with a trocar insertion, possibly close to an internal organ, which could be damaged if impaled by the trocar.

The procedure normally involves the surgeon viewing the interior of the abdominal wall via the laparoscope as the second trocar is inserted. Insertion involves pressure and twisting of the trocar in order to incise layers of tissue, and the final stages of insertion are heralded by peritoneal membrane tenting before the trocar tip breaches the abdominal wall and enters the abdominal cavity. At least in the latter stages of insertion, the surgeon will try to angle the trocar away from vulnerable organs, to avoid inadvertent perforation.

The procedure is relatively straightforward when the surgeon is experienced, the patient is of average size, and the surgeon is using new sharp trocars. However, in less experienced hands, with older tools, and small or obese patients, problems can be experienced.

It is an object of the present invention to provide a device and method for inserting a trocar in a surgical procedure so as to reduce the pressure needed to insert the trocar and to reduce potential damage to internal organs.

### SUMMARY OF THE INVENTION

A tool is provided which is adapted to be attached to the end of a laparoscopic telescope, to project ahead of the viewing lens of the telescope, the tool having a ring at its foremost end, which can be pushed into contact with the abdomen wall at a point where a trocar is to be inserted, for supporting the abdominal wall around the entry point of the trocar.

Typically the plane of the ring is angled relative to the axis of the telescope.

Typically the telescope has a 30° oblique visual field so that the ring can be positioned to as to appear in the top of the field of view, as seen by the surgeon through the telescope.

The force applied by the trocar on the skin is now more effective because it is concentrated over a smaller surface area due to the resistance presented to the surrounding skin by the presence of the ring. Once the trocar tip has penetrated the abdominal wall it enters the ring but is prevented from passing completely through the ring by selecting the diameter of the ring accordingly. By pushing the ring against the peritoneum the trocar tip will be prevented from penetrating significantly into the abdominal cavity

In use the trocar is first pushed gently against the abdomen where an incision is to be made to enable a tool to be inserted, and the region viewed from within by the surgeon. The ring is positioned on the inside of the abdominal wall adjacent to the peritoneum so that the ring surrounds the protrusion in the peritoneum caused by the pressure of the trocar against the external surface of the abdomen wall. The trocar is then rotated and forced inwardly in known manner to form the incision, the ring serving to resist significant inward movement of the trocar.

The invention also lies in a laparoscopic tool when fitted with a ring as aforesaid at the leading end thereof, so that whilst viewing the interior of the cavity using a laparoscopic telescope the ring attached to the leading end of the tool can be intruded through a previously made incision in the abdomen, to assist in the making of a further incision by another trocar, used in conjunction with the tool mounted ring.

In a preferred arrangement an elongate tool having a ring as aforesaid is fitted to the end of the laparoscopic telescope

The invention also lies in a method of inserting a tool to assist in laparoscopic surgery using a laparoscopic telescope, comprising the steps of:

1) anaesthetising the patient;

2) forming an umbilical opening in manner known per se;

3) inflating abdomen with gas via umbilical opening, or otherwise lifting abdomen;

4) fitting a shaft to protrude forward of the leading end of a laparoscopic telescope, with a trocar support ring at the leading end of the shaft;

5) inserting the ring and shaft and thereafter the telescope through the umbilical opening;

6) positioning a trocar against the outer surface of the abdomen and gently forcing the trocar so as to indent but not penetrate the skin;

7) illuminating the peritoneum on the inside of the abdomen wall and viewing same using the telescope until the protrusion in the peritoneum caused by the pointed end of the trocar is in the field of view;

8) moving the telescope so as to position the ring around the protrusion in the peritoneum;

9) gently forcing the trocar through the skin so that the tip of the trocar is received in and retained by the ring after penetration;

10) removing the trocar and inserting a tool through the incision formed by the trocar.

The invention also lies in a method of inserting a tool to assist laparoscopic surgery after a first trocar incision has been made in accordance with the foregoing method, comprising the further steps of:

1) Attaching a support ring to the leading end of the tool before it is inserted through the first incision (see step 10 above) or selecting a tool having such a ring at its leading end;

2) Introducing the ring ahead of the tool through the incision;

3) Viewing the interior of the abdominal cavity through the telescope;

4) Applying a trocar against another region of the abdomen so as to indent but not penetrate the peritoneum;

5) Locating the ring on the end of the tool so as to surround the protrusion in the peritoneum;

6) Gently forcing the trocar through the skin so that the tip of the trocar is received in and retained by the ring;

7) Removing the trocar and inserting a tool through the incision.

### DESCRIPTION OF THE DRAWINGS

FIG. 1 is a trocar support tool as seen from the top.

FIG. 2 shows the trocar support tool from the side.

FIG. 3 shows a view of the trocar support ring 1 as seen through the telescope.

FIG. 4 shows the trocar support tool attached to a laparoscopic telescope.

FIG. 5 shows a trocar support tool attached to a laparoscopic tool.

FIG. 6 shows a schematic representation of the peritoneum distortion preceding a trocar insertion as seen from a laparoscopic telescope without the use of the invention.

FIG. 7 shows the tip of the trocar breaking through the peritoneum (again without the use of the invention).

FIG. 8 is a schematic representation of a trocar insertion, with a trocar support positioned in accordance with the invention.

FIG. 9 shows the trocar after it has penetrated the peritoneal lining of the abdominal wall and entered the ring of the trocar support.

FIGS. 10 and 11 show to an enlarged scale how the ring co-operates with the protruding peritoneum.

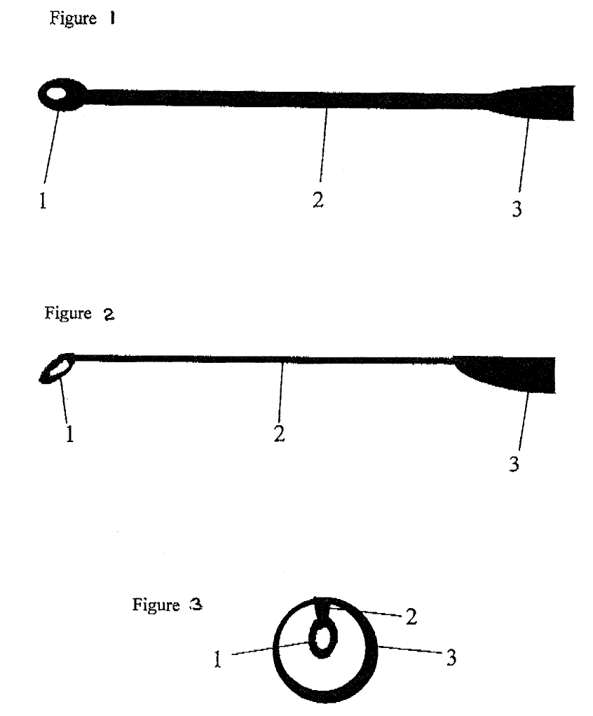

### DESCRIPTION OF THE FIGURES

Reference numeral 1 identifies a ring which is to be positioned on the inside of the abdominal wall to serve as a trocar support. Reference numeral 2 identifies the main shaft to which the trocar support ring is attached, and reference numeral 3 identifies an enlarged opposite end of the shaft, which is thereby adapted to be fitted to one end of the laparoscopic telescope.

The ring 1 is angled relative to the shaft 2 so that the ring is visible in the field of view of the telescope. This enables the trocar tip to be seen as it forces its way through the abdomen wall into the centre of the ring, (as will be illustrated in a later Figure).

The view of FIG. 3 shows where the ring appears in the field of view of the telescope.

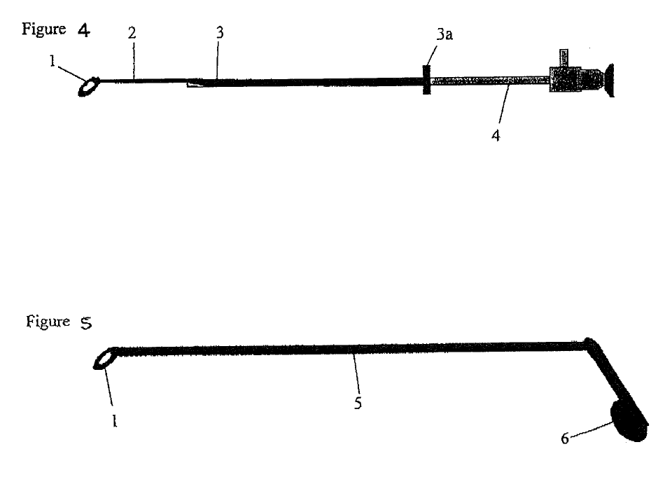

The complete assembly of tool and telescope is shown in FIG. 4, where the telescope (of known design) is denoted by reference numeral 4, The shaft 2, 3 is prevented from sliding off the telescope by means of a clamp or stop device 3a, which is not shown in the other Figures.

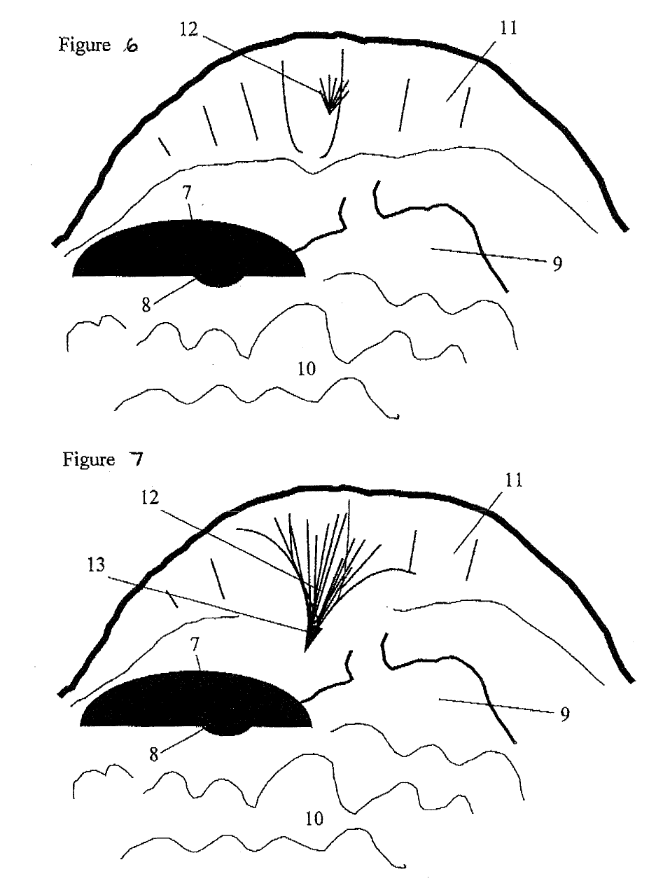

Alternatively the ring 1 may be attached to a shaft 5 of a laparoscopic tool having a handle 6, where it may be more convenient to insert the trocar support ring 1 separately from the telescope. Such a modified tool is shown in FIG. 5.

FIG. 6 shows the view as seen via the telescope as a trocar enters the abdomen. The telescope is assumed to enter via the umbilicus and to be looking up towards the head of the patient. The internal abdominal organs and structure are labelled with reference numbers as follows:

The gallbladder (8) lies in front of the liver (7).

The stomach (9) lies above the small intestine (10).

The peritoneal lining on the inside of the abdominal wall is denoted by (11) and this is seen being deformed at (12) to form a wigwam or tent-like shape by a trocar pressed against the skin from outside.

FIG. 7 shows schematically what is seen via a telescope positioned as described in relation to FIG. 6, after the tip (13) of the trocar has just penetrated the apex of the tented lining (12) of the peritoneum.

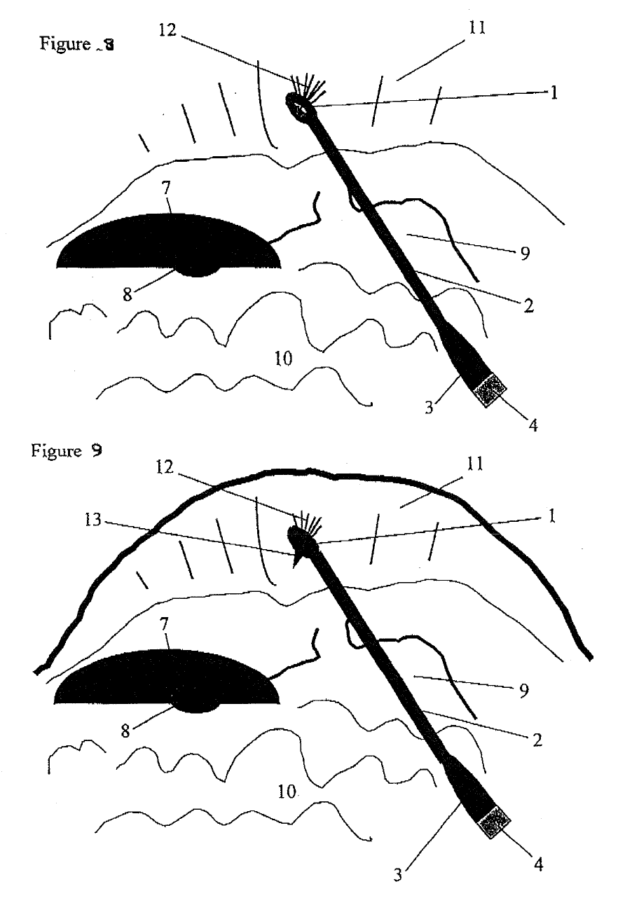

FIGS. 8 and 9 show how presence of a support ring 1 in accordance with the invention, supports the peritoneum around the tenting of the lining and initially provides a guide and support during the tenting step and thereafter a stop for the trocar, after penetration. In FIGS. 8 and 9, the ring 1 is shown at the end of a shaft 2 attached at 3 to the end of the telescope 4. However, it is to be understood that the ring 1 might instead be carried at the end of a shaft 5 of a tool having a handle 6 as shown in FIG. 5.

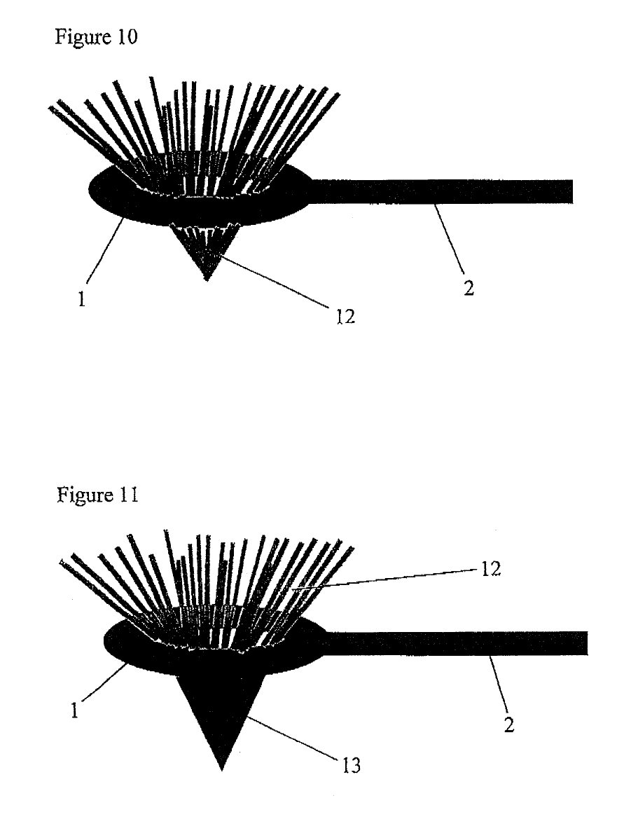

FIG. 10 illustrates the action of the support ring 1 and how it supports the peritoneum and defines the region which will become tented under the pressure of the trocar tip as at 12.

As the externally applied force exerted through the trocar is increased, the trocar tip 13 penetrates the lining as shown in FIG. 11, centrally of the ring 1. By carefully selecting the size of the ring 1, so that the trocar will not pass completely therthrough, the trocar is prevented by the ring 1 from entering deep into the abdominal cavity after the peritoneum has been perforated by the trocar. Thus the size of the ring can be chosen to prevent the trocar tip from entering too far into the abdominal cavity.

1. A laparoscopic instrument having a ring at its foremost end adapted to be pushed into contact with the abdomen wall at a point where a trocar is to be inserted, for supporting the abdominal wall around the entry point of the trocar.

2. An instrument as claimed in claim 1 wherein the plane of the ring is angled relative to the lengthwise axis of the instrument.

3. An instrument as claimed in claim 1 for use with a trocar wherein the diameter of the ring is selected so that it will admit the tip of the tocar but is too small to allow the trocar to pass completely therethrough, so that by pushing the ring against the peritoneum, the trocar will be prevented from penetrating significantly into the abdominal cavity.

4. An instrument as claimed in claim 1 further comprising clamp or stop means adapted to prevent the tool from leaving the instrument.

5. An instrument as claimed in claim 1 wherein the instrument is a laparoscopic telescope.

6. An instrument as claimed in claim 5 wherein the telescope has a 30° oblique visual field so that the ring can be positioned so as to appear in the top of the field of view when viewed through the telescope.

7. An attachment adapted to be fitted to a laparoscopic instrument, comprising a sleeve adapted for fitting to a leading end of the instrument, stop or clamp means for securing it to the instrument and a ring at its foremost end adapted to be pushed into contact with the abdomen wall at a point where a trocar is to be inserted, for supporting the abdominal wall around the entry point of the trocar.

8. A method of controlling the insertion of a trocar through the abdomen of a patient using a laparoscopic instrument as claimed in claim 1 wherein the trocar is first pushed gently against the abdomen where an incision is to be made, the region is viewed from within using a laparoscopic telescope, the ring is positioned against the peritoneum so that the ring surrounds the protrusion in the peritoneum caused by the pressure of the trocar against the external surface of the abdomen wall, and thereafter the pressure of the trocar on the skin is increased and the trocar rotated in known manner to form an incision, whilst firmly pressing the ring in the opposite sense to resist inward movement of the trocar beyond that permitted by the ring.

9. A method as claimed in claim 8 wherein the attachment is mounted on the telescope and the combination is pushed through a first incision and used to view the formation of a second incision in the peritoneum.

10. A method of inserting a tool to assist in laparoscopic surgery using a laparoscopic telescope, comprising the steps of:
1) anaesthetising the patient;
2) forming an umbilical opening in manner known per se;
3) inflating abdomen with gas via umbilical opening, or otherwise lifting abdomen;
4) fitting a shaft to protrude forward of the leading end of the laparoscopic telescope, with a trocar support ring at the leading end of the shaft;
5) inserting the ring and shaft and thereafter he telescope through the umbilical opening;
6) positioning a trocar against the outer surface of the abdomen and gently forcing the trocar so as to indent but not penetrate the skin;
7) illuminating the peritoneum on the inside of the abdomen wall and viewing same using the telescope until the protrusion in the peritoneum caused by the pointed end of the trocar is in the field of view;
8) moving the telescope so as to position the ring around the protrusion in the peritoneum;
9) gently forcing the trocar through the skin so that the tip of the trocar is received in and retained by the ring after penetration;
10) removing the trocar and inserting a tool through the incision formed by the trocar.

11. A method as claimed in claim 10 whereby a further tool is to be inserted wherein the following steps are performed:
1) attaching a support ring to the leading end of the further tool before it is inserted through the first incision, or selecting a tool having such a ring at its leading end;
2) introducing the ring ahead of the further tool through the incision;
3) viewing the interior of the abdominal cavity through the telescope;
4) applying a trocar against another region of the abdomen so as to indent but not penetrate the peritoneum;
5) locating the ring on the end of the further tool so as to surround the protrusion in the peritoneum;
6) gently forcing the trocar through the to make the incision so that the tip of the trocar is received in and prevented from continued movement into the abdominal cavity by the ring;
7) removing the trocar and inserting another tool through the incision.
